(19) United States
(12) Patent Application Publication
(10) Pub. No.: US 2024/0423884 A1
(43) Pub. Date: Dec. 26, 2024

(54) COSMETIC HAVING TITANIUM DIOXIDE IN THE AQUEOUS PHASE

(71) Applicant: BEIERSDORF AG, Hamburg (DE)

(72) Inventors: Jan NILSSON, Hamburg (DE); Berkay Daniel SIVACILAR, Hamburg (DE); Alexis GOULET-HANSSENS, Hamburg (DE); Magdalena VON WEDEL-PARLOW, Hamburg (DE)

(21) Appl. No.: 18/274,020

(22) PCT Filed: Jan. 14, 2022
(86) PCT No.: PCT/EP2022/050706

§ 371 Date: Jul. 25, 2023

(30) Foreign Application Priority Data
Feb. 2, 2021 (DE) 10 2021 200 944.1

(51) Int. Cl. A61K 8/29; A61K 8/06; A61K 8/27; A61K 8/73; A61Q 17/04

(52) U.S. Cl. CPC A61K 8/29; A61K 8/064; A61K 8/27; A61K 8/73; A61Q 17/04; A61K 2800/621; A61K 2800/623; A61K 2800/651

(57) ABSTRACT

Cosmetic preparation containing or consisting of an aqueous phase containing titanium dioxide particles and one or more gums (INCI: Gum) and the preparation thereof.

The present invention relates to a cosmetic preparation containing or consisting of an aqueous phase containing titanium dioxide particles and one or more gums (INCI: Gum) and the preparation thereof.

The trend away from pale complexion towards “healthy, athletic tanned skin” has continued for many years. To achieve this, people expose their skin to sunlight, since this causes pigment formation in the sense of melanin formation. However, the ultraviolet radiation of sunlight also has a harmful effect on skin. In addition to acute damage (sunburn), long-term damage such as an increased risk of developing skin cancer occurs on excessive irradiation with light from the UVB range (wavelength: 280-320 nm). Excessive exposure to UVB and UVA radiation (wavelength: 320-400 nm) additionally leads to a weakening of the elastic and collagen fibers in connective tissue.

This leads to numerous phototoxic and photoallergic reactions and results in premature aging of the skin.

To protect the skin, a series of photoprotective filter substances have therefore been developed which may be used in cosmetic preparations. These UVA and UVB filters are in most industrialized countries collated in the form of positive lists such as Annex 7 of the Kosmetikverordnung [German Cosmetics Ordinance]

The plethora of commercially available sunscreens must not however obscure the fact that these prior art preparations have a number of disadvantages.

For instance, a multiplicity of sunscreens are known from the prior art which comprise micronized titanium dioxide as UV filter. This is incorporated into the oil phase of the preparations because it would clump together in the water phase and cannot be evenly distributed in a sustained manner. However, there is a fundamental interest in being able to also stably incorporate titanium dioxides into the water phase, for example in order to be able to produce aqueous pigment-based sunscreens or to increase the UV protection of pigment-based sunscreen emulsions.

It was therefore the object of the present invention to find ways and means of being able to incorporate conventional UV filters based on titanium dioxide in a stable and uniformly finely distributed manner into a water phase. This water phase containing titanium dioxide should be suitable for emulsification with an oil phase.

Surprisingly, the object is achieved by a cosmetic preparation containing an aqueous phase or consisting of an aqueous phase containing titanium dioxide particles and one or more gums (INCI: Gum).

This aqueous preparation forms a flexible gel in which the titanium dioxide particles are stable and finely distributed. Surprisingly, there are also no titanium dioxide deposits on the vessel walls of the reactor vessel during production of the gel, which would entail extensive cleaning after production, as is usually the case in the prior art.

It is advantageous according to the invention if the titanium dioxide particles have a primary particle size between 5 and 200 nm, measured by transmission electron microscopy.

According to the invention, these titanium dioxide particles are advantageously coated with silica or an organopolysiloxane. The preferred embodiments according to the invention are characterized in that the titanium dioxide particles are coated with silica (silicon dioxide and/or silicic acid).

It is particularly preferable in accordance with the invention in this case if the titanium dioxide coated with silica has a layer of dimethicone, simethicone or methicone on the outer side of the silica layer (i.e., on the side facing away from the titanium dioxide). Of these substances, particular preference in accordance with the invention is given to dimethicone. Here, for example, the titanium dioxide “Parsol TX” from the company DSM is suitable. An example of organopolysiloxane-coated titanium dioxides is, for example, “Aeroxide TiO₂ T 805” from Evonik.

It is advantageous in the context of the present invention if the gums are selected from the group of the compounds gellan gum (INCI: Gellan Gum), sclerotium gum (INCI: Scletorium Gum) and xanthan gum (INCI: Xanthan Gum). According to the invention, preference is given to using gellan gum (INCI: Gellan Gum) and/or scletorium gum (INCI: Sclerotium Gum). Both high-acylated and low-acylated modifications of gellan gum may be used. The high-acylated gellan gums result in higher viscosity gels than the low-acylated gellan gums.

It is advantageous in the context of the present invention if the preparation contains titanium dioxide particles at a concentration of 0.1 to 20% by weight, based on the total weight of the water phase of the preparation. It is preferred according to the invention if the preparation contains titanium dioxide particles at a concentration of 5 to 20% by weight, based on the total weight of the water phase of the preparation.

The total weight of the water phase, also referred to as the combined water phases in the context of the present disclosure, is to be understood as the sum of water phase 1 and water phase 2 (see below).

Embodiments of the present invention that are advantageous according to the invention are characterized in that the preparation contains the gum(s) at a total concentration of 0.1 to 1% by weight, based on the total weight of the water phase of the preparation.

It is advantageous in the context of the present invention if the preparation contains glycerin at a concentration of 0.1 to 5% by weight, based on the total weight of the water phase of the preparation. In the preparations according to the invention, the addition of glycerol results in a softer gel being formed in the water phase.

It is also advantageous according to the invention when the preparation contains water at a concentration of 80 to 95% by weight, based on the total weight of the water phase of the preparation.

Embodiments of the present invention that are advantageous according to the invention are also obtained if the preparation contains phenoxyethanol at a concentration of 0.1 to 0.4% by weight, based on the total weight of the water phase of the preparation.

In one embodiment that is advantageous according to the invention, the preparation according to the invention may contain a citrate buffer in the water phase.

When using low-acylated gellan gum, the addition of citrate buffer results in higher-viscosity gels than without citrate buffer. When using high-acylated gellan gum, xanthan gum and sclerotium gum, the addition of citrate buffer results in lower-viscosity gels than without citrate buffer.

It is advantageous according to the invention when magnesium sulfate, calcium chloride and/or sodium chloride is added to the aqueous phase at a total concentration of 0.29 to 0.7% by weight, based on the total weight of the water phase of the preparation.

The cosmetic preparation according to the invention can be advantageously used according to the invention in two different product forms.

Firstly, it is in accordance with the invention if the preparation is in the form of an aqueous gel.

Secondly, it is in accordance with the invention if the preparation is in the form of an emulsion.

If the preparation according to the invention is in the form of an emulsion, it is particularly advantageous according to the invention if the preparation is in the form of a water-in-oil emulsion (W/O emulsion).

If the preparation according to the invention is in the form of an emulsion (in particular a W/O emulsion), it is advantageous according to the invention if the preparation contains zinc oxide. It is preferred according to the invention if the preparation contains the zinc oxide in the oil phase.

Advantageously in accordance with the invention, the preparation according to the invention can be used as a cosmetic sunscreen. In combination with the zinc oxide, highly effective purely pigment-based sunscreens can be produced in this way.

On the other hand, it is of course also possible to add chemical UV filters to the preparation, for example one or more UV filters selected from the group of the compounds 2-phenylbenzimidazole-5-sulfonic acid and/or salts thereof; phenylene-1,4-bis(2-benzimidazyl)-3,3′,5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl) sulfonic acid salts; 2,2′-methylenebis(6-(2H-benzotriazol-2-yl)-4-(1,1,3,3-tetramethylbutyl) phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene) camphor; 3-benzylidenecamphor; 4-(tert-butyl)-4′-methoxydibenzoylmethane; terephthalidenedicamphorsulfonic acid; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate; homomenthyl salicylate; 2-ethylhexyl 2-hydroxybenzoate; dimethicodiethyl benzalmalonate; 3-(4-(2,2-bis ethoxycarbonylvinyl) phenoxy) propenyl) methoxysiloxane/dimethylsiloxane copolymer; dioctylbutylamidotriazone (INCI: Diethylhexyl Butamidotriazone); 2,4-bis {[4-(2-ethylhexyloxy)-2-hydroxy]phenyl}-6-(4-methoxyphenyl)-1,3,5-triazine; 2,4-bis [5-1(dimethylpropyl)benzoxazol-2-yl-(4-phenyl)imino]-6-(2-ethylhexyl)imino-1,3,5-triazine with CAS No. 288254-16-0; tris(2-ethylhexyl) 4.4′,4″-(1,3,5-triazine-2,4,6-triyltriimino)trisbenzoate (also: 2,4,6-tris [anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; piperazine derivatives.

It is also preferred in accordance with the invention if the preparation is free from mineral oil, paraffin wax, microcrystalline wax, shellac wax and polyethylene wax, free from polyacrylates, crosslinked acrylate/C10-C30 alkyl acrylate crosspolymers and vinylpyrrolidone/hexadecene copolymers, and free from 3-(4-methylbenzylidene) camphor, 2-hydroxy-4-methoxybenzophenone (INCI: Oxybenzone), 2-ethylhexyl 4-methoxycinnamate (INCI: Octylmethoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

In addition, the preparation according to the invention may comprise the customary lipophilic ingredients known for preparations of this kind, for example oils, fats, waxes and the like.

Moreover, it is advantageous in accordance with the invention if the preparation contains one or more compounds selected from the group of the compounds glycyrrhetic acid, urea, arctiin, alpha-lipoic acid, folic acid, phytoene, D-biotin, coenzyme Q10, hyaluronic acid, alpha-glucosylrutin, carnitine, carnosine, caffeine, natural and/or synthetic isoflavonoids, glycerylglucose, creatine, creatinine, taurine, β-alanine and/or licochalcone A, panthenol, tocopherol, tocopherol acetate, vitamin C, vitamin C derivative, Glycyrrhiza inflata root extract, polidocanol, magnolol, honokiol, tocopheryl acetate, dihydroxyacetone; 8-hexadecene-1,16-dicarboxylic acid, glycerylglucose, (2-hydroxyethyl) urea, vitamin E and derivatives thereof, hyaluronic acid and/or salts thereof, licochalcone A.

Last but not least, embodiments of the present invention that are advantageous in accordance with the invention are characterized in that the preparation contains one or more alcohols selected from the group of the compounds ethanol, 1,2-propylene glycol, 1,2-butylene glycol, 1,3-butylene glycol, 1,2-pentanediol, 1,2-hexanediol, 1,2-octanediol, 1,2-decanediol, 2-methyl-1,3-propanediol, phenoxyethanol and/or ethylhexylglycerin, preference being given in accordance with the invention to the use of ethanol, 1,2-pentanediol, 1,2-hexanediol, 1,2-octanediol, 1,2-decanediol, 2-methyl-1,3-propanediol, phenoxyethanol and/or ethylhexylglycerin. An alternative preferred embodiment with alcohols is the combination of 1,2-propylene glycol, 1,2-butylene glycol and/or 1,3-butylene glycol with 1,2-octanediol.

The water phase of the emulsion according to the invention may comprise customary auxiliaries.

Also in accordance with the invention is a method for the preparation of such a cosmetic preparation which is characterized in that the gum is dissolved in a first water phase (water phase 1) with stirring, the titanium dioxide is suspended in a second water phase (water phase 2) with stirring and then the two water phases are combined with stirring.

In this method, it is advantageous according to the invention if the water phases 1 and 2 are prepared and the two water phases are combined at a temperature of 70° C. to 80° C. and the combined water phases are then cooled to room temperature with stirring.

When using gellan gum, the desired gel formation occurs particularly rapidly.

The addition of magnesium sulfate or calcium chloride also leads to more rapid gel formation, in contrast to sodium chloride.

Embodiments of the method according to the invention that are advantageous according to the invention are characterized in that water phase 2 is homogenized using an UltraTurrax and water phase 1 and the combined water phases are homogenized using a stirrer.

It is also advantageous for the method according to the invention if the glycerin is added to water phase 1.

Advantageous embodiments of the method according to the invention are characterized in that the phenoxyethanol is added to the combined water phases.

If the preparations containing citrate buffer are to be prepared, the method according to the invention is advantageously characterized in that sodium hydroxide (NaOH) is added to water phase 1 after the addition of gum and glycerin, and citric acid is added to the combined water phases.

Preferably in accordance with the invention, the citric acid is added at the end of the process, i.e., when cooling the preparation.

In addition, embodiments of the method that are advantageous according to the invention are then characterized in that sodium hydroxide and the citric acid are each used in an amount of 0.1 to 1.0% by weight, based on the total weight of the water phase.

The method according to the invention can also be used to prepare the emulsions according to the invention. In such a case, it is advantageous according to the invention if the combined water phases are combined with an oil phase while stirring.

In such a case, the method according to the invention, advantageously according to the invention, is characterized in that the combined water phases are combined with an oil phase with stirring, with heating of the combined water phases and the oil phase to a temperature of 70-90° C. prior to combination.

Last but not least, a preparation prepared by the method according to the invention is also in accordance with the invention.

### EXAMPLES

The examples below are intended to illustrate the present invention without limiting it.

### Method A: pH Treatment

I. Water phase 1: The gum according to the invention is slowly added to half the amount of the water phase, which also already contains the glycerin, with stirring (magnetic stirrer, 300-500 rpm) slowly at 75° C. and the mixture is stirred for ca. 20 minutes until the gum has completely dissolved. Subsequently, 0.9-1.0% by weight of NaOH solution is added dropwise and the mixture is stirred for a further 15 minutes at 75° C.
II. Water phase 2: The other half of the water phase is heated separately to 70 to 75° C. with stirring, and the titanium dioxide is incorporated into this second half of the water phase using a Turrax (model T25 or T50) at 10,000 rpm. After 5-7 minutes, this water phase is added to water phase 1 at 75° C. with stirring.
III. Combined water phases 1 and 2: The combined water phases 1 and 2 are stirred at 75° C. for a further 30 minutes using a magnetic stirrer (300-500 rpm) and ca. 0.4% by weight of phenoxyethanol is added. After stirring for a further 5 minutes, 0.9 to 1.0% by weight of citric acid is slowly added and this total water phase is cooled with stirring.

At about 50° C. the gel phase visibly begins to form. The formation of the gel phase is complete when room temperature is reached.

Comment: In the case of gellan gum (both high-acylated and low-acylated gellan gum) up to 1% by weight of salts (NaCl, CaCl₂), MgSO₄) may be added during the cooling process to accelerate gel formation. The gel is formed more rapidly using bivalent salts (CaCl₂), MgSO₄) than with NaCl.

### Method B: Without pH Treatment

I. Water phase 1: The gum according to the invention is slowly added to half the amount of the water phase, which also already contains the glycerin, with stirring (magnetic stirrer, 300-500 rpm) at 75° C. and the mixture is stirred for ca. 20 minutes until the gum has completely dissolved.
II. Water phase 2: The other half of the water phase is heated separately to 70 to 75° C. with stirring, and the titanium dioxide is incorporated into this second half of the water phase using a Turrax (model T25 or T50) at 10,000 rpm. After 5-7 minutes, this water phase is added to water phase 1 at 75° C. with stirring.
III. Combined water phases 1 and 2: The combined water phases 1 and 2 are stirred at 75° C. for a further 30 minutes using a magnetic stirrer (300-500 rpm) and ca. 0.4% by weight of phenoxyethanol is added. This entire water phase is then cooled with stirring.

At about 50° C. the gel phase visibly begins to form. The formation of the gel phase is complete when room temperature is reached.

Comment: In the case of gellan gum (both high-acylated and low-acylated gellan gum) up to 1% by weight of salts (NaCl, CaCl₂), MgSO₄) may be added during the cooling process to accelerate gel formation. The gel is formed more rapidly using bivalent salts (CaCl₂), MgSO₄) than with NaCl.

### Formulations

| | Example | Example | Example | Example | Example | Example |
|---|---|---|---|---|---|---|
| INCI | 1 | 2 | 3 | 4 | 5 | 6 |
| Aqua | 89.48 | 90.36 | 89.33 | 89.36 | 90.13 | 89.32 |
| Gellan gum | 0.27 | — | — | — | — | — |
| (low-acetylated) | | | | | | |
| Gellan gum | — | 0.27 | — | 0.13 | — | — |
| (high- | | | | | | |
| acetylated) | | | | | | |
| Xanthan gum | — | — | 0.45 | 0.26 | 0.45 | 0.22 |
| Sclerotium gum | — | — | — | — | — | 0.22 |
| Phenoxyethanol | 0.40 | 0.40 | 0.40 | 0.4 | 0.4 | 0.4 |
| Sodium | 0.89 | — | 0.89 | 0.89 | — | 0.89 |
| Hydroxide | | | | | | |
| Titanium | 8.95 | — | 8.93 | 8.93 | — | — |
| dioxide + Silica + | | | | | | |
| Dimethicone | | | | | | |
| Titanium | — | 8.97 | — | — | 9.01 | 8.93 |
| dioxide + Silica | | | | | | |
| Citric Acid | Approx. 0.89 | — | Approx. 0.89 | Approx. 0.89 | — | Approx. 0.89 |

| INCI | Example 7 | Example 8 | Example 9 |
|---|---|---|---|
| Aqua | 89.95 | 89.95 | 89.95 |
| Gellan gum | 0.27 | 0.27 | 0.27 |
| (high-acetylated) | | | |
| Phenoxyethanol | 0.41 | 0.41 | 0.41 |
| Titanium dioxide + Silica | 9.1 | 9.1 | 9.1 |
| NaCl | 0.27 | — | — |
| CaCl₂ | — | 0.27 | — |
| MgSO₄ | — | — | 0.27 |

1.-26. (canceled)

27. A cosmetic preparation, wherein the preparation comprises an aqueous phase or consists of an aqueous phase that contains titanium dioxide particles and one or more gums.

28. The preparation of claim 27, wherein the titanium dioxide particles have a primary particle size of from 5 nm to 200 nm, measured by transmission electron microscopy.

29. The preparation of claim 27, wherein the titanium dioxide particles are coated with silica or an organopolysiloxane.

30. The preparation of claim 27, wherein the one or more gums comprise one or more of gellan gum, sclerotium gum and xanthan gum.

31. The preparation of claim 27, wherein the preparation comprises the titanium dioxide particles at a concentration of from 0.1% to 20% by weight, based on a total weight of a water phase of the preparation.

32. The preparation of claim 27, wherein the preparation comprises the one or more gums at a total concentration of from 0.1% to 1% by weight, based on a total weight of a water phase of the preparation.

33. The preparation of claim 27, wherein the preparation further comprises glycerin at a concentration of from 0.1% to 5% by weight, based on a total weight of a water phase of the preparation.

34. The preparation of claim 27, wherein the preparation comprises water at a concentration of from 80% to 95% by weight, based on a total weight of a water phase of the preparation.

35. The preparation of claim 27, wherein the preparation further comprises phenoxyethanol at a concentration of from 0.1% to 0.4% by weight, based on a total weight of a water phase of the preparation.

36. The preparation of claim 27, wherein the aqueous phase of the preparation comprises a citrate buffer.

37. The preparation of claim 27, wherein magnesium sulfate, calcium chloride and/or sodium chloride are present in the aqueous phase at a total concentration of from 0.29% to 0.7% by weight, based on a total weight of a water phase of the preparation.

38. The preparation of claim 27, wherein the preparation is in the form of an aqueous gel.

39. The preparation of claim 27, wherein the preparation is in the form of an emulsion.

40. The preparation of claim 27, wherein the preparation is in the form of a water-in-oil emulsion (W/O emulsion).

41. The preparation of claim 27, wherein the preparation further comprises zinc oxide.

42. The preparation of claim 41, wherein the preparation comprises the zinc oxide in an oil phase.

43. A method for preparing the cosmetic preparation of claim 27, wherein the method comprises dissolving gum in a first water phase (water phase 1) with stirring, suspending the titanium dioxide in a second water phase (water phase 2) with stirring, and then combining water phase 1 and water phase 2 with stirring.

44. The method of claim 43, wherein the two water phases are combined at a temperature of 70° C. to 80° C. and the combined water phases are then cooled to room temperature with stirring.

45. The method of claim 43, wherein water phase 2 is homogenized using an UltraTurrax and water phase 1 and the combined water phases are homogenized using a stirrer.

46. A preparation prepared by the method of claim 43.
